(19) United States
(12) Patent Application Publication
(10) Pub. No.: US 2007/0129784 A1
(43) Pub. Date: Jun. 7, 2007

(54) Stents

(75) Inventors: Andreas Lendlein, Berlin (DE); Peter Simon, Aachen (DE); Karl Kratz, Berlin (DE); Birgit Schnitter, Ulm (DE)

Correspondence Address: DAVIS WRIGHT TREMAINE, LLP, 2600 CENTURY SQUARE, 1501 FOURTH AVENUE, SEATTLE, WA, 98101-1688, US

(73) Assignee: MNEMOSCIENCE GMBH, Ubach-Palenberg (DE)

(21) Appl. No.: 10/560,452

(22) PCT Filed: Jun. 9, 2004
(86) PCT No.: PCT/EP04/06262

§ 371 Date: Jun. 14, 2006

(30) Foreign Application Priority Data
Jun. 13, 2003 (DE) 103 26 779.4
Jun. 13, 2003 (DE) 103 26 781.6
Dec. 10, 2003 (DE) 103 57 742.4
Dec. 10, 2003 (DE) 103 57 743.2

(51) Int. Cl. A61F 2/84; A61F 2/94

(52) U.S. Cl. 623/1.11; 623/1.19; 623/1.38

(57) ABSTRACT

The present invention refers to stents for use in the non-vascular and vascular field, which comprise an SMP material.

The subject matter of the invention is a temporary stent made from shape memory polymers (SMP) for use in the non-vascular or vascular field. The stent can be minimized by the shape memory effect and it may be removed by minimally invasive surgery. A further subject matter of the invention is a method of implanting and removing the stent and for manufacturing and programming the stent.

### PRIOR ART

To treat clogged vessels or constricted tubular organs or after surgical procedures, tubular tissue supports (stents) are inserted into the tubular organ. They serve for keeping open the constriction portion or for taking over the function of the injured tubular organ to re-enable normal passage or discharge of body liquids. Stents are also inserted into the blood vessel to treat clogged or constricted blood vessels, said stents keeping open the constricted portion and re-enabling normal blood flow.

Stents are usually cylindrical structures made of a kind of wire netting (wire coil design) or tubes, which may be perforated or which may not be perforated (slotted tube design). Conventional stents have a length of 1 and 12 cm and may have a diameter of 1 to 12 mm.

The mechanical demands on a stent are contradictory. On the one hand, a stent must exert high radial forces onto the tubular organ to be supported. On the other hand it is required that the stent can be radially compressed to be able to easily insert it into a tubular organ without injuring the vessel wall or the surrounding tissue.

This problem was solved in that the stents are inserted in compressed form and are mounted only after having reached the correct position. In the compressed state the diameter is smaller than in expanded state. This process can basically also be used for the minimal invasive removal of the stent. A possible problem is, however, that the metallic materials usually used do not always completely regularly expand and cannot be folded again, which is a potential risk of injury for the bordering tissue.

For the minimal invasive insertion of a stent, two different technologies have established (market report “US peripheral and vascular stent and AAA stent graft market” (Frost & Sullivan), 2001):
Balloon expandable stents (system consists of balloon, catheter, stent)
Self-expandable stent (system consists of a sleeve for insertion (protective sheeth), catheter, stent);

Self-expanding stents consist of a shape memory material (SM material), wherein metallic SM materials, such as nitinol come in the fore. The shape memory effect is an effect that has been examined during the past years with great interest, which enables an aimed change of shape by applying an outer stimulus (regarding details in this respect, reference is made to the already published literature, e.g. “Shape Memory Alloys”, Scientific American, Vol. 281 (1979), pages 74 to 82). The materials are able to specifically change their shape in the case of an increase in temperature. The shape memory effect is activated to increase the diameter of stents “automatically” and to fix them at the location where they are used.

The removal of expanded stents is problematic, as was already indicated above. If the stent must be pulled out of a tubular cavity, there is a risk of injuring the surrounding tissue by abrasion, because the stent is too large and has sharp edges. The shape memory effect is therefore also applied to reduce the diameter of the stent if a stent must be removed again. Examples for removable implants (stents) made of shape memory metals are known from the prior art: U.S. Pat. No. 6,413,273 “Method and system for temporarily supporting a tubular organ”; U.S. Pat. No. 6,348,067 “Method and system with shape memory heating apparatus for temporarily supporting a tubular organ”, U.S. Pat. No. 5,037,427 “Method of implanting a stent within a tubular organ of a living body and of removing same”; U.S. Pat. No. 5,197,978 “Removable heat-recoverable tissue supporting device”.

Nitinol cannot be used in the case of a nickel allergy. The material is also very expensive and can only be programmed by laborious methods. This programming methods need comparatively high temperatures so that a programming within the body is not possible. The SM material is therefore programmed outside the body, i.e. it is brought to its temporary shape. After implantation, the shape memory effect is activated and the stent expands, i.e. it regains its permanent shape. A removal of a stent by again utilizing the shape memory effect is then not possible. A frequent problem in metallic stents not only in the vascular area is above that the occurrence of a restenosis.

Other metallic stents of SM materials, such as from U.S. Pat. No. 5,197,978 on the other hand enable a utilization of the shape memory effect to remove the stent. However, these metallic materials are very laborious to manufacture, and the tissue compatibility is not always ensured. Due to the inadequately adapted mechanical properties of the stents, inflammations and pain often occur.

The temporary stent described in U.S. Pat. No. 5,716,410 “Temporary stent and method of use” is a coil made of a shape memory plastic material. The SMP material has an embedded heating wire. The heating wire is connected via a catheter shaft to an electrical controller, wherein the shaft end being a hollow tube is put over the end of the coil. If the implanted stent is heated, which is in its expanded, temporary shape, above the switching temperature Tₜᵣₐₙₛ, the diameter of the coil reduces. This shall enable a simple removal of the stent. A disadvantage of the coil structure is that the radial forces are too low to expand the tubular cavities. The radial forces of the coil spread only over a small contact surface to the tissue. There is even a risk of a local mechanical overload by pressure, possibly by incision into the tissue. Moreover, the attachment of the catheter shaft (heating element) to the heating wire of the implanted coil proves to be difficult, since the catheter shaft must only be put over the one end of the coil.

Further examples of the prior art refer to stents of shape memory polymers, which can be implanted in compressed, temporary shape, wherein the desired permanent size is generated by the shape memory effect at the place of use (U.S. Pat. No. 4,950,258, U.S. Pat. No. 6,245,103, U.S. Pat. No. 6,569,191, EP 1033145). The removal of the stent is implemented either by a further surgical operation or by the resorption of the material in the body. A disadvantage of the materials used is their embrittlement when they resorb and the generation of particles that may lead to cloggings when released from the device. Moreover, a resorption may also change the structure/nature of an implant such that an incompatibility with blood and/or tissue occurs.

Further problems that often occur are pain caused by the insufficient mechanical adaptation of the stent to the surrounding tissue and the displacement of the stent.

### OBJECT OF THE INVENTION

Since stents have increasingly captured an extending field of use in medicine, endeavors must be made to overcome the above-mentioned disadvantages. Thus, stents for the non-vascular or vascular use are needed which enable a minimal invasive implantation and at the same time enable the gentle removal thereof. The materials for the stent shall above that be adaptable to the respective place of use, e.g. in view of varying mechanical loads. The materials shall preferably enable a further functionalization of the stent, e.g. by embedding further medically useful substances.

To overcome disadvantages of the prior art, the following is required:
a simple procedure which enables the minimally invasive implantation and removal of a stent,
a stent, which can be removed minimally invasively and atraumatically, preferably by using the shape memory effect,
a stent, which when used vascularly or non-vascularly does not grow into the vessel wall,
a stent which has a surface that is haemocompatible,
a stent, which during use has a sufficient mechanical strength/integrity so that the function is not affected despite a possibly occurring biodegradation,
a stent that does not grow together with the tissue to be supported so that it can easily be removed, and which also inhibits the formation of a bio-film or the encapsulation of germs,
a method of manufacturing and programming such a stent.

### SHORT DESCRIPTION OF THE INVENTION

This object is solved by the subject matter of the present invention, as it is defined in the claims. These stents comprise a shape memory material (SMP material), preferably an SMP material, which reveals a thermally induced or light-induced shape memory effect. The SMP materials to be used according to the invention may remember one or two shapes.

Stents of this type solve the above-mentioned problems, either on the whole or at least partially. Thus, the present invention provides stents, comprising an SMP material, which can be used minimally invasively and atraumatically by the use of the shape memory effect, which are tissue-compatible and which have a sufficient strength/stability so that they can be removed after the desired time of use during which they exert their function without loss of mechanical stability.

Particularly to prevent the generation of a bio-film and to prevent growing-in, the stent may be modified for the non-vascular use, by a suitable selection of segments of the SMP material, by a surface modification, particularly a micro-structuring, or by suitable coating, or by the use of disinfecting substances that are released by the stent after implantation.

Furthermore, the stent, depending on the location of use, may be adapted to the respective demands by suitable modifications, since for instance different pH-conditions, the existence of specific enzymes or generally the microbiological environment may make special demands. By the respective selection of segments for the SMP-materials, these demands can be taken into consideration.

### SHORT DESCRIPTION OF THE FIGURES

FIG. 1 schematically shows the difference in size between the permanent and the temporary shape of the stent of the invention.

FIG. 2 shows a schematical view of the working steps for introducing and for removing the stent. The bright grey part shows the stent, the dark grey part shows the balloon of the catheter and the black part shows the catheter.

FIG. 3 schematically shows the functional principle of a stent with two shapes in the memory.

### DETAILED DESCRIPTION OF THE INVENTION

In preferred embodiments, the object is solved by a stent of SMP, characterized in that
the stent in its permanent shape is pre-mounted onto a temperature-controlled balloon catheter or onto a balloon catheter equipped with a suitable light source (particularly UV),
the diameter of the temporary shape is larger than in the permanent shape (cf.

FIG. 1),
the temporary shape acts as a tissue support,
the SMP has a switching temperature of 40° C. and higher or a switching wavelength of 260 nm or more,
the implanted stent takes the permanent, compressed shape caused by the SM effect, so that it can easily be removed by minimal invasive surgery.

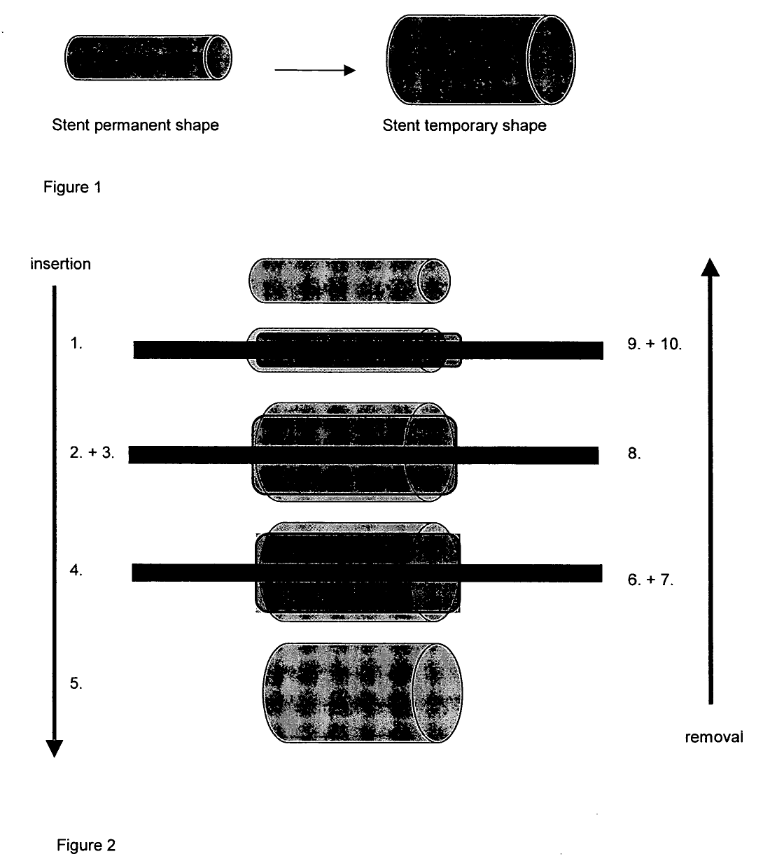

A possible procedure for the minimal invasive insertion and removal of a stent, comprises the following steps (FIG. 2):

Insertion:
1. The stent pre-mounted onto a temperature-controlled balloon catheter or a balloon catheter equipped with a suitable light source is inserted into the tubular, non-vascular organ in a minimal invasive manner,
2. the placed stent is heated possibly by means of a catheter above its Trans (at least 40° C.) (the balloon fills up with warm water or gas),
3. the stent is brought to the temporary shape (expanded) in that the balloon catheter is further pumped up with warm water or gas until it has reached the desired shape/expansion, i.e. the stent is only programmed directly at the implantation location.
4. the expanded stent is cooled down by means of the catheter below Ttrans (the balloon fills up with cold water or gas) or is irradiated by light with a wavelength greater than 260 nm to fix the temporary shape,
5. the balloon is contracted and/or the irradiation is stopped and the balloon catheter is removed.

Removal:
1. For removal, the balloon catheter is inserted into the stent portion,
2. the balloon is expanded by liquid (water) or gas to produce a direct contact with the stent and to ensure the heat transport or the irradiation with light,
3. the stent is heated over Ttrans by means of the catheter or it is irradiated with light of a wavelength smaller than 260 nm to activate the shape memory effect, to bring the stent back to its permanent (smaller) shape,
4. the balloon is slowly relieved (discharge of liquid (water) or gas), wherein the stent contracts (SM effect) and automatically fixes itself on the balloon,
5. the compressed stent is possibly cooled down and is removed together with the balloon catheter.

As an alternative, this procedure can also be described as follows:

Insertion:
1. The stent pre-mounted on a temperature-controlled balloon catheter is inserted into the tubular organ by means of minimal invasive surgery,
6. the placed stent is heated by means of the catheter over its Tₜᵣₐₙₛ (at least 40° C.) (the balloon fills up with warm water or gas),
7. the stent is brought into the temporary shape (expanded) in that the balloon catheter is further pumped up with warm water or gas, until it has reached the desired shape/expansion; i.e. the stent is directly programmed at the implantation location,
8. the expanded stent is cooled down by means of the catheter below Tₜᵣₐₙₛ (the balloon fills up with cold water or gas) to fix the temporary shape,
9. the balloon is contracted and the balloon catheter is removed:

Removal:
10. For removal the balloon catheter is inserted into the stent portion,
11. the balloon is expanded by liquid (water) or gas to generate a direct contact with the stent and to ensure the heat transport,
12. the stent is heated by means of the catheter over Tₜᵣₐₙₛ (the balloon fills up with warm water or gas) to activate the shape memory effect, to bring the stent back into its permanent (smaller) shape,
13. the balloon is slowly released (discharge of liquid (water) or gas), wherein the stent contracts (SM effect) and automatically fixes itself on the balloon,
14. the compressed stent is possibly cooled down and is removed together with the balloon catheter.

A possible method for the minimal invasive insertion and removal of a stent with light-induced shape memory comprises the following steps (FIG. 2):

Insertion:
1. The stent pre-mounted on a balloon catheter equipped with a suitable light source is inserted into the tubular organ by means of minimal invasive surgery,
2. the stent is brought to the temporary shape (expanded) in that the balloon catheter is further pumped up with (warm) water or gas until it has reached the desired shape/expansion; i.e. the stent is directly programmed at the implantation location,
3. the expanded stent is irradiated by light having a wavelength greater than 260 nm to fix the temporary shape,
4. the balloon is contracted and/or the irradiation is stopped and the balloon catheter is removed.

Removal:
5. for removal, the balloon catheter is inserted into the stent portion,
6. the balloon is expanded by liquid (water) or gas to produce a direct contact with the stent and to ensure the irradiation with light,
7. the stent is irradiated by light having a wavelength less than 260 nm to activate the shape memory effect, to bring the stent back it is permanent (smaller) shape,
8. the balloon is slowly relieved (discharge of liquid (water) or gas), wherein the stent contracts (SM effect) and automatically fixes itself on the balloon,
9. the compressed stent is removed together with the balloon catheter.

It is especially preferred in this connection, if the stents, which are only programmed at the location of use, since they are only there brought into the temporary shape, are heated outside the body over their transition temperature before insertion into the body. Since forces do not act on the stent at this point, a change of the expansion of the stent does not take place. However, this heating enables that the SMP material of the stent becomes elastic and flexible. In this manner, the pre-heated stents can be inserted better and more easily compared to the rather rigid stents before heating. Particularly if large stents are used and/or stents that must be pushed through heavily wound vessels or the like, this pre-heating offers a significant improvement regarding the insertion of the stent.

In many applications in which stents are placed, it is very important that the actual position of the stent exactly corresponds to the desired location of use. This is particularly important if two stents are inserted in series, since a precise placing is then particularly important to ensure the desired success. In the case of conventional stents, a correction of the placing of the stents is, however, hard to achieve, since a further folding of the stent at the location of use is problematic. The stents according to the invention, which are only programmed directly at the location of use offer a significant advantage. Since the stents according to the invention in this embodiment in their expanded form exist in temporary condition, a simple reduction of the stent can be achieved by activating the SM effect so that the stent reduced again can be placed again, which enables a simple correction of the placing. After the correction, the stent according to the invention is then newly programmed again by the method steps described above and is left in the temporary state as tissue support.

The insertion with correction can be outlined by the following method steps:
1. The stent pre-mounted on a temperature-controlled balloon catheter is inserted into the tubular organ.
2. The placed stent is heated over the transition temperature by means of the catheter.
3. The stent is brought into the temporary shape (expanded) until it has reached the desired shape (expansion).
4. The expanded stent is cooled down below the transition temperature by means of the catheter and thus fixed in the temporary state.

If it is detected after that the stent is not yet correctly placed, the following correction steps are additionally carried out:
5. The stent is heated above the transition temperature by means of the catheter to activate the shape memory effect and to bring the stent back to is smaller shape.
6. The balloon is slowly relieved, wherein the stent contracts.
7. The stent sitting on the balloon can no be placed correctly.

Subsequently, steps 3 and 4 are repeated to newly place the stent. Subsequently, the catheter is removed.

The correction procedure described here can of course analogously also be carried out with the shape memory materials which show a light-inducted shape memory effect.

Stents with Two Shapes in the Memory

A stent programmed twice has the advantage that it can first of all be implanted in compressed form by minimal invasive surgery and its fixing at the location of use is carried out by heating. The first change in shape (e.g. diameter enlargement) is carried out. After the desired dwelling time at the location of use, the stent may be removed by means of minimal invasive surgery in that it is heated again to cause the second change of shape (e.g. diameter reduction).

Stents with two shapes in the memory can be made of SMP which are characterized by covalent net points and two switching segments or two transitional temperatures Tₜᵣₐₙₛ, wherein Tₜᵣₐₙₛ 1<Tₜᵣₐₙₛ 2 applies and both switching temperature lie above body temperature. The covalent net points determine the permanent shape of the stent, the switching segments each determine a temporary shape.

In an embodiment, a stent in the form of a tube is characterized in that the diameter of the permanent shape Dₚₑᵣₘ is small, the diameter of the first temporary shape Dₜₑₘₚ 1 is larger than Dₚₑᵣₘ and the diameter of the second temporary shape Dₜₑₘₚ 2 is smaller than Dₜₑₘₚ 1: Dₚₑᵣₘ<Dₜₑₘₚ 1>Dₜₑₘₚ2.

The second temporary shape may have an identical diameter or it may deviate from the permanent shape: Dₚₑᵣₘ=Dₜₑₘₚ2 or Dₚₑᵣₘ≠Dₜₑₘₚ2.

The double programming of the stent is constitutes of the following method steps:
1. Heating the stents above Tₜᵣₐₙₛ 2,
2. expansion of the stent below Tₜᵣₐₙₛ2 and above Tₜᵣₐₙₛ1
3. cooling below Tₜᵣₐₙₛ2 and above Tₜᵣₐₙₛ1,
4. compression of the stent to Dₜₑₘₚ1,
5. cooling below Tₜᵣₐₙₛ1.

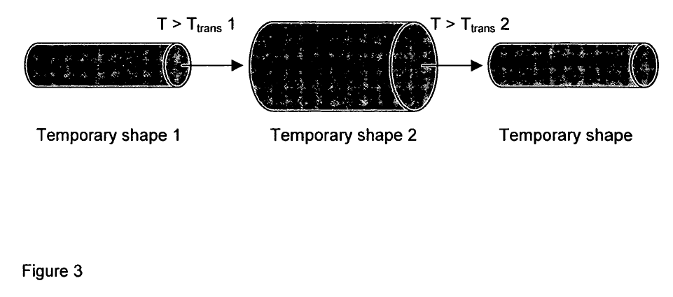

When heating the stent programmed twice above Tₜᵣₐₙₛ1, the shape of Dₜₑₘₚ 1 changes to Dₜₑₘₚ2, i.e. the diameter enlarges. When further heating above Tₜᵣₐₙₛ2, Dₚₑᵣₘ is taken, i.e. the diameter reduces again (FIG. 3).

The invention will now be described further.

The stent of the present invention comprises an SMP material. Thermoplastic materials, blends and networks are suitable. Composite materials of SMP with inorganic nano particles are also suitable. Preferably, a heating element is not embedded into the SMP material. The shape memory effect can be activated thermally by means of a heatable medium, by applying IR or NIR irradiation, by applying an oscillating electrical field or by UV irradiation.

The definition that the stent according to the invention comprises an SMP material shall define that the stent on the one hand substantially consists of an SMP material, but that on the other hand the stent may also be a conventional stent, embedded or coated with an SMP material. These two essential constructions offer the following advantages.

Stents, which essentially consist of SMP materials, use the SMP material to determine the mechanical properties of the stents. By the fact that the materials, which will now be described, are used for this purpose, a favorable tissue compatibility is ensured. Furthermore, such stents, as described above, may be implanted and removed by minimal invasive surgery. The SMP materials may also be relatively easily processed, which facilitates manufacture. Finally, the SMP materials can be compounded or layered with further substances so that a further functionalization is possible. In this connection, reference is made to the following statements.

The second embodiment that is possible in principle is a stent, which comprises a conventional basic frame, such as a “wire netting structure” or a deformable tube. These basic frames are coated by an SMP material or they are embedded therein. Particularly wire netting constructions proved that the SMP materials may exert a sufficiently great power to deform the basic frame if the shape memory effect is activated. This embodiment therefore allows to combine the positive properties of the conventional stents with the above-mentioned positive effects of the SMP materials. Particularly, stents with a very high mechanical resistance can thereby be obtained, since the conventional basic frame contributes to this. Thus, this embodiment is particularly suitable for stents that are exerted to high mechanical loads.

The surface of the stent is compatible in view of the physiological environment at the place of use, by suitable coating (e.g. hydrogel coating) or surface micro-structuring. In the stent design the basic conditions such as the pH value or the number of germs must be taken into consideration depending on the location of use.

Suitable materials for the stents of the present invention will now be described.

SMP materials in the sense of the present invention are materials, which are capable, due to their chemical-physical structure, to carry out aimed changes in shape. Besides their actual permanent shape the materials have a further shape that may be impressed on the material temporarily. Such materials are characterized by two structural features: network points (physical or covalent) and switching segments.

SMP with a thermally induced shape memory effect have at least one switching segment with a transitional temperature as switching temperature. The switching segments form temporary cross linking portions, which resolve when heated above the transitional temperature and which form again when being cooled. The transitional temperature may be a glass temperature T_{g} of amorphous ranges or a melting temperature Tₘ of crystalline ranges. It will now in general be designated as Tₜᵣₐₙₛ. At this temperature the SMP show a change in shape.

Above Tₜᵣₐₙₛ the material is in the amorphous state and is elastic. If a sample is heated above the transitional temperature Tₜᵣₐₙₛ, deformed in the flexible state and then cooled down below the transitional temperature, the chain segments are fixed by freezing degrees of freedom in the deformed state (programming). Temporary cross linking portions (non-covalent) are formed so that the sample cannot return to its original shape also without external load. When re-heating to a temperature above the transitional temperature, these temporary cross linking portions are resolved and the sample returns to its original shape, By re-programming, the temporary shape can be produced again. The accuracy at which the original shape is obtained again is designated as resetting ratio.

In photo-switchable SMP, photo-reactive groups, which can reversibly be linked with one another by irradiation with light, take over the function of the switching segment. The programming of a temporary shape and re-generation of the permanent shape takes place in this case by irradiation without a change in temperature being necessary.

Basically, all SMP materials for producing stents can be used. As an example, reference can be made to the materials and the manufacturing methods, which are described in the following applications, which by reference directly belong to the content of the application on file:

German patent applications: 10208211.1, 10215858.4, 10217351.4, 102173050.8, 10228120.3, 10253391.1, 10300271.5, 10316573.8.

European patent applications: 99934294.2, 99908402.3

SMP materials with two shapes in the memory are described in the U.S. Pat. No. 6,388,043 which is comprised herewith by reference.

Conventional materials for stents, which can be used within the framework of the present invention particularly in the above-mentioned second embodiment, are as follows:

Bio-stable materials fundamentally suitable for the use on the medical sector are polyethylene (PE), polypropylene (PP), polyethylene terephthalate (PET), PVC polycarbonate (PC), polyamide (PA), polytetrafluoroethylene (PTFE), polymethacrylate, polymethylmethacrylate (PMMA), polyhydroxyethylm ethacrylate (PHEMA), polyacrylate, polyurethane (PUR), polysiloxane, polyetheretherketone (PEEK), polysulphone (PSU), polyether, polyolefines, polystyrene.

Materials that are already established for the use in non-vascular areas are e.g. polysiloxane (catheter and tube probes, bladder prostheses), PHEMA (urinary bladder prostheses) and PA (catheter tubes).

Materials that are already established for the use in the vascular area are e.g. PUR (artificial blood vessels, heart valves), PET (artificial blood vessels, blood vessel coatings), PA (mitral valves), polysiloxanes (heart valves), PTFE (vessel implants).

To manufacture the stents according to the invention, thermoplastic elastomers can be used. Suitable thermoplastic elastomers are characterized by at least two transitional temperatures. The higher transitional temperature can be assigned to the physical network points which determine the permanent shape of the stent. The lower transitional temperature at which the shape memory effect can be activated can be associated to the switching segments (switching temperature, Tₜᵣₐₙₛ). In the case of suitable thermoplastic elastomers the switching temperatures are typically approximately 3 to 20° C. above the body temperature.

Examples for thermoplastic elastomers are multiblockcopolymers. Preferred multiblockcopolymers are composed of the blocks (macrodioles) consisting of α,ω diol polymers of poly(e-caprolacton) (PCL), poly(ethylene glycol) (PEG), poly(pentadecalacton), poly(ethyleneoxide), poly(propyleneoxide), poly(propylene glycol), poly(tetrahydrofuran), poly(dioxanon), poly(lactide), poly(glycolid), poly(lactide-ranglycolid), polycarbonates and polyether or of α,ω, diol copolymers of the monomers on which the above-mentioned compounds are based, in a molecular weight range Mₙ of 250 to 500,000 g/mol. Two different macrodiols are linked by the aid of a suitable bi-functional coupling reagent (especially an alipathic or aromatic diisocyanate or di-acid chloride or phosgene) to form a thermoplastic elastomer with molecular weights Mₙ in the range of 500 to 50,000,000 g/mol. In a phase-segregated polymer, a phase with at least one thermal transition (glass or melt transition) may be associated in each of the blocks of the above-mentioned polymer irrespective of the other block.

Multiblockcopolymers of macrodiols on the basis of pentadeclaracton (PDL) and—caprolacton (PCL) and a diisocyanate are especially preferred. The switching temperature—in this case a melting temperature—may be set over the block length of the PCL in the range between approx. 30 and 55° C. The physical network points to fix the permanent shape of the stent are formed by a second crystalline phase with a melting point in the range of 87 to 95° C. Blends of multiblockcopolymers are also suitable. The transitional temperature can be set in an aimed manner by the mixing ratio.

To manufacture the stents according to the invention, polymer networks can also be used. Suitable polymer networks are characterized by covalent network points and at least one switching element with at least one transitional temperature. The covalent network points determine the permanent shape of the stents. In the case of suitable polymer networks, the switching temperature, at which the shape memory effect can be activated, are typically approximately 3 to 20° C. above the body temperature.

To produce a covalent polymer network, one of the macrodiols described in the above section is cross linked by means of a multifunctional coupling reagent. This coupling reagent may be an at least tri-functional, low-molecular compound or a multi-functional polymer. In the case of a polymer, it might be a star polymer with at least three arms, a graft polymer with at least two side chains, a hyper-branched polymer or a dendritic structure. In the case of the low-molecular and the polymer compounds, the final groups must be able to react with the diols. Isocyanate groups may especially be used for this purpose (polyurethane networks).

Amorphous polyurethane networks of trioles and/or tetroles and diisocyanate are especially preferred. The representation of the star-shaped pre-polymers such as oligo[(raclactate)-co-glycolat]triol or -tetrol is carried out by the ring-opening copolymerization of rac-dilactide and diglycolide in the melt of the monomers with hydroxy-functional initiators by the addition of the catalyst dibutyl tin(IV)oxide (DBTO). As initiators of the ring-opening polymerization, ethylene glycol, 1,1,1-tris(hydroxy-methyl)ethane or pentaerythrit are used. Analogously, oligo(lactat-co-hydroxycaproat)tetroles and oligo(lactate-hydroxyethoxyacetate) as well as [oligo(propylene glycol)-block-oligo(raclactate)-co-glycolat)]triole are manufactured. The networks according to the invention may simply be obtained by conversion of the pre-polymers with diisocyanate, e.g. an isomeric mixture of 2,2,4- and 2,4,4-trimethylhexane-1,6-diisocyanate (TMDI), in solution, e.g. in dichloromethane, and subsequent drying.

Furthermore, the macrodiols described in the above section may be functionalized to corresponding α,ω-divinyl compounds, which can thermally or photo-chemically be cross linked. The functionalization preferably allows a covalent linking of the macro-monomers by reactions that do not result in side products. This functionalization is preferably provided by ethylenic unsaturated units, particularly preferred acrylate groups and methacrylate groups, wherein the latter are particularly preferred. In this case the conversion to α,ω-macrodimethacrylates or macrodiacrylates by reaction with the respective acid chlorides in the presence of a suitable base may particularly be carried out. The networks are obtained by cross linking the end group-functionalized macro-monomers. This cross linking may be achieved by irradiation of the melt, comprising the end group-functionalized macromonomer component and possibly a low-molecular co-monomer, as will be explained further below. Suitable method conditions for this are the irradiation of the mixture in melt, preferably at temperatures in the range of 40 to 100° C., with light of a wavelength of preferably 308 nm. As an alternative, a heat cross linking is possible if a respective initiator system is used.

If the above-described macromonomers are cross linked, networks are produced having a uniform structure, if only one type of macromonomers is used. If two types of monomers are used, networks of the AB-type are obtained. Such networks of the AB-type may also be obtained if the functionalized macromonomers are copolymerized with suitable low-molecular or oligomer compounds. If the macro-monomers are functionalized with acrylate groups or methacrylate groups, suitable compounds, which can be copolymerized, are low-molecular acrylates, methacrylates, diacrylates or dimethacrylates. Preferred compounds of this type are acrylates, such as butylacrylate or hexylacrylate, and methacrylates such as methylmethacrylate and hydroxyethylmethacrylate.

These compounds, which can be copolymerized with the macromonomers, may exist in a quantity of 5 to 70 percent by weight related to the network of macromonomer and the low-molecular compound, preferably in a quantity of 15 to 60 weight percent. The installation of varying quantities of the low-molecular compound takes place by the addition of respective quantities of compound to the mixture to be cross linked. The installation of the low-molecular compound into the network takes place at a quantity that corresponds to that of the cross linking mixture.

The macromonomers to be used according to the invention will now be described in detail.

By variation of the molar weight of the macrodiols, networks with different cross linking densities (or segment lengths) and mechanical properties can be achieved. The macromonomers to be cross linked covalently preferably have a numeric average of the molar weight determined by GPC analysis of 2000 to 30000 g/mol, preferably 500 to 20000 g/mol and particularly preferred of 7500 to 15000 g/mol. The macromonomers to be covalently cross linked preferably have on both ends of the marcomonomer chain a methacrylate group. Such a functionalization allows the cross linking of the macromonomers by simple photo-initiation (irradiation).

The marcomonomers are preferably bio-stable or very slowly degradable polyester macromonomers, particularly preferably polyester macromonomers on the basis of—carprolacton or pentadeclaracton. Other possible polyester macromonomers are based on lactide units, glycolide units, p-dioxane units and the mixtures thereof and mixtures with—caprolacton units, wherein polyester macromonomers with caprolacton units or pentadecalacton units are particularly preferred. Preferred polyester macromonomers are furthermore poly(caprocacton-co-glycolide) and poly(caprolacton-co-lactide). The transitional temperature can be set through the quantity ratio of the co-monomers. Especially preferred are also biostable macromonomers on the basis of polyethers, polycarbonates, polyamides, polystyrene, polybutyleneterephthalate and polyethylene terephthalate.

Particularly preferred are the macromonomers polyester, polyether or polycarbonates to be used according to the invention, comprising the linkable end groups. An especially preferred polyester to be used according to the invention is a polyester on the basis of—caprolacton or pentadecalacton, for which the above-mentioned statements about the molar weight apply. The manufacture of such a polyester macromonomer, functionalized at the ends, preferably with methacrylate group, may be manufactured by simple syntheses, that are known to the person skilled in the art. These networks, without consideration of the further essential polymer components of the present invention, show semi-crystalline properties and have a melting point of the polyester component (determinable by DSC measurements) that depends on the type of polyester component used and which is also controllable thereby. As is known, this temperature (Tₘ1) for segments based on caprolacton units is between 30 and 60° C. depending on the molar weight of the macromonomer.

A preferred network having a melt temperature as switching temperature is based on the macromonomer poly(caprolacton-co-glycolide)-dimethacrylate. The macromonomer may be converted as such or may be co-polymerized with n-butylacrylate to form an AB-network. The permanent shape of the stent is determined by covalent network points. The network is characterized by a crystalline phase, whose melting temperature can be set e.g. by the comonomer ratio of caprolacton to glycolide in an aimed manner in the range of 20 to 57° C. n-butylacrylate as comonomer may e.g. be used for optimizing the mechanical properties of the stent.

A further preferred network having a glass temperature as switching temperature is obtained from an ABA tri-blockdimethylacrylate as macromonomer, characterized by a central block B of polypopyleneoxide and end blocks A of poly(rac-lactide). The amorphous networks have a very broad switching temperature range.

To manufacture stents with two shapes in the memory, networks having two transitional temperatures are suitable, such as inter-penetrating networks (IPNs). The covalent network is based on poly(caprolacton)-dimethacrylate as macromonomer; the inter-penetrating component is a multiblockcopolymer of macrodiols based on pentadecalacton (PDL) and—caprolacton (PCL) and a diisocyanate. The permanent shape of the material is determined by the covalent network points. The two transitional temperatures—melt temperatures of the crystalline phases—may be utilized as switching temperatures for a temporary shape. The lower switching temperature Tₜᵣₐₙₛ may be set via the block length of the PCL in the range between approx. 30 and 5° C. The upper switching temperature Tₜᵣₐₙₛ2 lies in the range of 87 to 95° C.

The above described SMP materials are substantially based on poly or oligoester segments. These SMP materials therefore partially reveal an insufficient stability in physiological environment, since the ester bonds can relatively simply be decomposed hydrolytically, although the stability is sufficient for most applications, particularly in stents that do not remain at the place of use for a very long period of time. Problems of this kind can, however, be overcome in that the SMP materials instead comprise segments on the basis of poly or oligoether units or poly or oligocarbonate units.

Segments of this kind may for instance be based on poly(ethyleneoxide), poly(propyleneoxide) or poly(tetramethyleneoxide).

To manufacture the stents according to the invention, photosensitive networks can also be used. Suitable photosensitive networks are amorphous and are characterized by covalent network points, which determine the permanent shape of the stent. A further feature is a photo-reactive component or a unit reversibly switchable by light, which determines the temporary shape of the stent.

In the case of the photosensitive polymers a suitable network is used, which includes photosensitive substituents along the amorphous chain segments. When being irradiated with UV light, these groups are capable of forming covalent bonds with one another. If the material is deformed and irradiated by light of a suitable wavelength λ1, the original network is additionally cross-linked. Due to the cross-linking a temporary fixing of the material in deformed state is achieved (programming). Since the photo-linking is reversible, the cross linking can be released again by further irradiation with light of a different wavelength λ2 and thus the original shape of the material can be reproduced again (reproduction). Such a photo-mechanical cycle can be repeated arbitrarily often. The basis of the photo-sensitive materials is a wide meshed polymer network, which, as mentioned above, is transparent in view of the irradiation intended to activate the change in shape, i.e. preferably forms an UV-transparent matrix. Networks of the present invention on the basis of low-molecular acrylates and methacrylates, which can radically be polymerized are preferred according to the invention, particularly C1-C6-meth(acrylates) and hydroxyderivatives, wherein hydroxyethylacrylate, hydroxyporpylmethacrylate, poly(ethyleneglycole)methacrylate and n-butylacrylate are preferred; preferably n-butylacrylates and hydroxyethylmethacrylate are used.

As a co-monomer for producing the polymer network of the present invention a component is used, which is responsible for the cross linking of the segments. The chemical nature of this component of course depends on the nature of the monomers.

For the preferred networks on the basis of the acrylatemonomers described above as being preferred, suitable cross linking agents are bi-functional acrylate compounds, which are suitably reactive with the starting materials for the chain segments so that they can be converted together. Cross linking agents of this type comprise short, bi-functional cross linking agents, such as ethylenediacrylate, low-molecular bi- or polyfunctional cross linking agents, oligomer, linear diacrylate cross linking agents, such as poly(oxyethylene)diacrylates or poly(oxypropylene)diacrylates and branched oligomers or polymers with acrylate end groups.

As a further component the network according to the invention comprises a photo-reactive component (group), which is also responsible for the activation of the change in shape that can be controlled in an aimed manner. This photo-reactive group is a unit which is capable of performing a reversible reaction caused by the stimulation of a suitable light irradiation, preferably UV radiation (with a second photo-reactive group), which leads to the generation or resolving of covalent bonds. Preferred photo-reactive groups are such groups that are capable of performing a reversible photodimerization. As a photo-reactive component in the photosensitive networks according to the invention, different cinnamic acid esters (cinnamates, CA) and cinnamylacylic acid ester (cinnamylacylates, CAA) can preferably be used.

It is known that cinnamic acid and its derivatives dimerize under UV-light of approx. 300 nm by forming cyclobutane. The dimeres can be split again if irradiation is carried out with a smaller wavelength of approx. 240 nm. The absorption maximum can be shifted by substituents on the phenyl ring, however they always remain in the UV range. Further derivatives that can be photodimerized, are 1.3-diphenyl-2-propene-1-on (chalcon), cinnamylacylic acid, 4-methylcoumarine, various orthos-substituted cinnamic acids, cinammolyxysilane (silylether of the cinnamon alcohol).

The photo-dimerization of cinnamic acid and similar derivatives is a [2+2] cyclo-addition of the double bonds to a cyclobutane derivative. The E-isomers as well as the Z-isomers are capable of performing this reaction. Under irradiation the E/Z-isomerization proceeds in competition with the cyclo-addition. In the crystalline state the E/Z-isomerization is, however inhibited. Due to the different possibilities of arrangement of the isomers with respect to each other, 11 different stereo-isomeric products (truxill acids, truxin acids) are theoretically possible. The distance of the double bonds of two cinnamic acid groups to one another required for the reaction is approximately 4 Å.

The networks are characterized by the following properties:

On the whole, the networks are favorable SMP materials, with high reset values, i.e. the original shape is also obtained in the case of running through a cycle of changes in shape several times at a high percentage, usually above 90%. A disadvantageous loss of mechanical property values does not occur.

To increase the haemocompatibility, the chemical structure of the SMP-materials used according to the invention can be modified, e.g. by the installation of the above-mentioned poly or oligoether units.

Processing of the Polymers to Become Stents

To process the thermoplastic elastomers to form stents, e.g. in the form of a hollow tube or the like (FIG. 1) all conventional polymer-technical methods such as injection molding, extrusion, rapid prototyping etc. can be used that are known to the person skilled in the art. Additionally, manufacturing methods such as laser cutting can be used. In the case of thermoplastic elastomers, different designs can be realized by spinning in mono and multi-filament threads with subsequent interweaving to a cylindrical network with a mesh structure.

In the manufacture of stents of polymer networks it must be taken care that the form in which the cross linking reaction of the macromonomers takes place corresponds to the permanent shape of the stent (casting method with subsequent curing). Especially the network materials according to the invention require, for further processing, special milling and cutting methods. The perforation or the cutting of a tube by the aid of LASER light of a suitable wavelength is suggested. By the aid of this technology—especially in the case of a combination of CAD and pulsed CO₂ or YAG lasers—shapes up to a size of 20 μm can be worked down without the material being exposed to a high thermal load (and thus undesired side reactions on the surface). As an alternative, a chip removing processing to obtain a ready stent is suggested.

The second embodiment is obtained by coating or embedding a conventional material (see above) into an SMP material by a suitable method.

The required mechanical properties of the stent depend on the place of use and require an adapted design. If the implanted stent is exposed to strong mechanical deformations, a very high flexibility is required without the stent collapsing during the movements. Basically, the “wire coil design” is more suitable. In other areas of organs that are located deeper the stent is less loaded mechanically by deformations but rather by a relative high external pressure. A stent suitable for this purpose must be characterized by high radial forces onto the ambient tissue. In this case the “slotted tube design” seems to be more suitable. Tubes with perforations enable the inflow of liquid from the ambient tissue into the stent (drainage).

Particularly the prior art often revealed problems in the blood vessels with small diameters, since the known stents are not flexible and adaptable enough for such vessels. However, the stents of the present invention also enable a safe application in such vessels, since the superior elastic properties of the SMP materials, i.e. the high elasticity at small deflections and high strength at a large expansion, protects the vessel for instance in the case of pulsatile movements of the arteries.

Since drainage effects are in the fore in the case of stents that shall be used on the non-vascular area, particularly a design with embedded conventional basic frame is favorable for such stents, or a design basically consisting of SMP material (perforated tube or network body), since in these designs the permeability for liquids necessary for the drainage is very simple while at the same time revealing a sufficient mechanical strength.

Functionalization of the Stents

For a more convenient insertion of the stent, this stent may possibly be provided with a coating which increases slippage (e.g. silicones or hydrogels).

Further possibilities of improving haemocompatibility comprise the possibility that a coating is provided (the materials necessary for this purpose are known to the person skilled in the art), or a micro-structuring of the surface can be made. Suitable methods of surface modification are for instance the plasma-polymerization and graft polymerization.

To localize the stent more easily by visual diagnostic procedures, the shape memory plastic material can be screened by a suitable x-ray contrast agent (e.g. BaSO₄). A further possibility can be seen in the installation of metal threads (e.g. stainless steel) into the stent. These metal threads do not serve stabilization purposes (but localization purposes); it is their only object to increase the x-ray contrast. A third possibility is seen in the screening with metals, which besides their high x-ray contrast also have virostatic, fungicidal or bactericidal properties (e.g. nano silver). A further alternative in this respect is the installation of x-ray opaque chromophores such as triiodine benzene derivatives into the SMP-materials themselves.

In a further embodiment, the SMP may be compounded with inorganic nano-particles. Examples are particles made of magnesium or magnesium alloys or magnetite. Particles made of carbon are also suitable. SMP functionalized in this way may be heated in an oscillating electrical field to active the shape memory effect.

The stent according to the invention may also be charged with a number of therapeutically effective substances, which support the healing process, which suppress the restenosis of the stent or which also prevent subsequent diseases. The following may especially be used:
anti-inflammatory active substances (e.g. ethacridine lactate)
analgetic substances (e.g. acetylsalicyclic acid)
antibiotic active substances (e.g. enoxacine, nitrofurantoin)
active substances against viruses, fungi (e.g. elementary silver)
antithrombic active substances (e.g. MS, clopidogel, hirudin, lepirudin, desirudin)
cytostatic active substances (e.g. sirolimus, rapamycin or rapamune)
immunosuppressive active substances (e.g. ABT-578)
active substances for lowering the restenosis (e.g. taxol, paclitaxel, sirolimus, actinomycin D).

The stent according to the invention can be charged with active substances in different ways.

The active substances can either be directly screened with the plastics or they may be attached onto the stent as a coating.

Stents of this kind may also be used in the field of genetic therapy.

If the material of the stent is directly screened with the active substances, the active substance can be released either in a degradation-controlled manner or in a diffusion-controlled manner. In the case of the degradation-controlled release the diffusion speed of the active substance from the matrix is slower than the degradation speed of the polymer. If this is the case, the active substance is advantageously embedded either into a degradable coating, which surrounds the stent or directly into the polymer material. In the case of the diffusion-controlled release, the diffusion speed of the active substance from the matrix is faster than the degradation speed of the polymer. In this the active substance is permanently discharged by the matrix.

As a third possibility the active substance may be introduced into the pores of a porous shape memory plastic material. After charging with the active substance the pores of the material are closed and the stent is brought to the effective location as described above. By a suitable external stimulus (heat or irradiation of light) the pores are opened and the active substance is abruptly released. For these application a shape memory plastic material is particularly suitable, which has shapes in the memory; in this case one of the shapes is responsible for the change in shape of the stent, the second shape is responsible for the opening of the pores.

If the active substances are introduced into the material of the stent according to the invention, the release of the active substances takes place after the stent was implanted. The release of the active substance involves the degradation of the stent; thus, it must be taken care that the diffusion speed of the active substance from the stent must be lower than the degradation speed of the material of the stent, and that the mechanical stability of the stent is not affected by this degradation.

In such embodiments, the stent may for instance comprise several SMP materials, e.g. one for safeguarding the stability/integrity of the stent and one coated on the surface of the stent and containing the active substances.

The following applications are especially possible:

Iliac Stents

These stents have a length of 10 to 120 mm, usually 40 to 60 mm. They are used in the abdominal area. Usually, two stents are used, since the use of long stents is difficult. The stents of the present invention are, however, characterized by a favorable flexibility and enable a very gentle minimal invasive application and removal, so that the stents of the present invention can also be used on lengths that are considered not to be feasible in the prior art.

Renal Stents

In this case a high radial strength is required, due to high elastic load in the kidney artery, which possibly requires an increased mechanical reinforcement of the stent. In this case either the “slotted tube design” is suitable or the use of conventional stents coated by or embedded into the SMP material. Both embodiments allow the use of radio-opaque markers. In this case it is furthermore important to ensure a safe installation of the stent on the balloon of the catheter and a precision during insertion. Due to the different anatomy of all creatures, adapted, variable lengths and diameters are required. Furthermore, the combination with a distal protective device and a plaque filter is advisable.

Carotid Artery Stents
A long stent can be used in this case to avoid the former technique of combination of two stents.
It can also be used at vessel bifurcations
Optimal adaptation to different diameters is possible
Networks with tight meshes are desirable and realizable (see above), because of filter function which is possibly required for avoiding the introduction of blood clots into the cerebrum (plaque filter function)
The stent must be pressure-stable, pressure could possibly be built up externally, the stent should not collapse.

Femoral-Poplietal Stents (Hip-Knee)

High radial strength due to high elastic load in the blood vessel, which possibly requires an increased mechanical reinforcement. In this case the “slotted tube design” is rather suitable (possibly by using a conventional framework), particularly the use of two long stents is conceivable.

Coronal Stents
wire coil design
atraumatic introduction without abrasive effects is an indispensable condition and possible with the stents of the present invention.

Design of Non-Vascular Stents

The essential fields of application are the entire gastrointestinal tract, trachea and esophagus, bile duct, ureter, urethra and oviduct. Accordingly, stents in various sizes are used. The different pH values of the body liquids and the occurrence of germs must individually be taken into consideration in the stent design.

Independent of the location of use, non-vascular stents are substantially used for the drainage of body liquids such as bile juice, pancreas juice or urine. Thus, the design of a perforated hose is advisable, which on the one hand may safely discharge the liquid to be discharged from the cavity, but which on the other hand absorbs the liquid across the entire way. Furthermore, the polymer material used must have a high flexibility to ensure wearing comfort. For a better identification in x-ray examinations, the starting material may be screened by x-ray contrast substances such as barium sulfate, or x-ray opaque chromphores are integrated into the SMP materials, e.g. by polymerization of suitable monomers. If stents are to be used in fields in which germs occur, the integration of antibiotic active substances into the material might be sensible.

The encrustation of the stents frequently occurring particularly in the uretheral area can be reduced by suitable coating or surface modification.

Fixing of the stent substantially depends on the location of use. In the case of a uretheral stent, the proximal end is located in the renal pelvis, the distal end is located in the urinary bladder or also outside of the body. The proximal end forms a loop after termination of the expansion in the renal pelvis and therefore ensures a safe hold.

Another possibility of fixing the stent is that the stent is tightly pressed to the surrounding tissue via radial forces towards the outside, or that it contains anchoring elements serving for fixing.

In the case of bile or kidney stents, an atraumatic placing and removal is an indispensable condition. It must particularly be ensured during placing that the tissue is not injured by abrasive effects thus causing inflammations. A stent used in this area does not have any retaining elements that could injure the tissue.

Suitable materials that are for instance suitable of being used in the present invention will now be stated as an example:

Examples for Multiblockcopolymers

The multiblockcopolymer was manufactured from macrodiols on the basis of pentadecalacton (PDL) and -caprolacton (PCL) and a diisocyanate. PDL defines the portion of pentadecalacton in the multiblockcopolymer (without consideration of the diisocyanate bridges) as well as the molecular weight of the polypentadecalacton segments. PCL defines the respective data for caprolacton units.

| | | | Molecular | | |
|---|---|---|---|---|---|
| | | | weight Mₙ of | E-module | Tensile |
| Exam- | | | the polyester | (70° C./ | strength |
| ple | PDL | PCL | urethane | MPa) | (MPa) |
| 1 | 100 percent | | 192000 | 17 | 18 |
| | by weight/ | | | | |
| | 10000 g/mol | | | | |
| 2 | 22 percent | 78 percent | 120000 | 1.4 | 5 |
| | by weight/ | by weight/ | | | |
| | 10000 g/mol | 10000 g/mol | | | |
| 3 | 41 percent | 59 percent | 196000 | 3 | 10 |
| | by weight/ | by weight/ | | | |
| | 10000 g/mol | 10000 g/mol | | | |
| 4 | 60 percent | 40 percent | 176000 | 7 | 8 |
| | by weight/ | by weight/ | | | |
| | 10000 g/mol | 10000 g/mol | | | |
| 5 | 80 percent | 20 percent | 185000 | 8.5 | 7 |
| | by weight/ | by weight/ | | | |
| | 10000 g/mol | 10000 g/mol | | | |
| 6 | 40 percent | 60 percent | 86000 | 3.5 | 4.5 |
| | by weight/ | by weight/ | | 35 (RT) | 23 (RT) |
| | 2000 g/mol | 4000 g/mol | | | |
| 7 | 50 percent | 50 percent | 75000 | 1.5 | 1.6 |
| | by weight/ | by weight/ | | 70 (RT) | 24 (RT) |
| | 3000 g/mol | 10000 g/mol | | | |
| 8 | 40 percent | 60 percent | 62000 | 3 | 9 |
| | by weight/ | by weight/ | | 45 (RT) | 30 (RT) |
| | 3000 g/mol | 10000 g/mol | | | |

The mechanical properties depending on the temperature for example 8 are as follows:

| | Breaking | | Tensile |
|---|---|---|---|
| T | strain | E-module | strength |
| (° C.) | (%) | (MPa) | (MPa) |
| 22 | 900 | 45 | 30 |
| 37 | 1000 | 25 | 30 |
| 50 | 1000 | 12 | 20 |
| 55 | 1050 | 7 | 15 |
| 60 | 1050 | 3 | 10 |
| 65 | 1000 | 3 | 10 |
| 70 | 1000 | 3 | 9 |
| 75 | 1000 | 3 | 7 |
| 80 | 1000 | 1.5 | 3 |

Examples for Polymer Networks

Suitable polymer networks are obtained by copolymerisation of a macrodimethacrylate, on the basis of glycolide units and □-caprolacton units with n-butylacrylate. The weight proportion of glycolide in the macrodimethylacrylate is 9 percent by weight (or 11 percent by weight in example 13). The molecular weights of the macrodimethacrylates are aproximately 10000 to 11000 g/mol.

| | Percent by weight butylacrylate | E- | |
|---|---|---|---|
| | in the network | module | Breaking strain |
| example | Determined by 13C-NMR | (MPa) | % |
| 9 | 17 | 11 | 271 |
| 10 | 28 | 8.1 | 422 |
| 11 | 41 | 6.4 | 400 |
| 12 | 56 | 6.5 | 399 |
| 13 | 18 | 8.8 | 372 |

Example for Amorphous Polymer Networks

The amorphous networks were manufactured from ABA triblockdimethacrylates, wherein A stands for segments of poly(rac-lactide) and B stands for segments of atactic poly(propyleneoxide) (Mₙ=4000 g/mol).

| | Mₙ [H-NMR] | | | | | |
|---|---|---|---|---|---|---|
| | ABA triblock- | Percent | | | Degree of | PD [GPC] |
| | dimethacrylate | by weight | T_{g}1 | T_{g}2 | methacrylation | ABA-triblock- |
| Example | (g/mol) | A | (DSC) (° C.) | (DSC) (° C.) | (%) ** | diole |
| 14 | 6400 | 38 | * | * | 77 | 1.4 |
| 15 | 6900 | 42 | 10 | 36 | 100 | 1.1 |
| 16 | 8000 | 50 | −41 | — | 64 | 1.3 |
| 17 | 8500 | 53 | −50 | 19 | 56 | 1.7 |
| 18 | 8900 | 55 | −59 | 16 | 99 | 1.4 |
| 19 | 10300 | 61 | −60 | 1 | 115 | 2.3 |

PD = Polydispersity
* Sample polymerized in the DSC-measurement
** values above 100 are to be ascribed to impurities

The polymer amorphous networks were examined in view of their further thermal and mechanical properties. The results of these examinations are combined in the following tables.

| | | | | Breaking | Rupture |
|---|---|---|---|---|---|
| | | | E-module | strain bei | strain at |
| | T_{g}1 | T_{g}2 | at 22° C. | 22° C. | bei 22° C. |
| example | (° C.) | (° C.) | (MPa) | (%) | (MPa) |
| 14 | −51 | 7 | 1.24 | 128 | 1.43 |
| 15 | −60 (−43*) | 4 (11*) | 2.02 | 71 | 0.94 |
| 16 | −46 | n.d. | 1.38 | 218 | 2.18 |
| 17 | −50 | 15 | 4.17 | 334 | 5.44 |
| 18 | −59 (−45*) | 7 (33*) | 4.54 | 110 | 1.89 |
| 19 | −62 (−49*) | 29 (43*) | 6.37 | 210 | 3.92 |

*determined by DMTA;
n.d.—not detectable

| | | Reset ratio | Temperature interval | Start temperature | Final temperature |
|---|---|---|---|---|---|
| | Shape | after 5 | of the transition | of the transition | of the transition |
| Example | fixing (%) | cycles (%)* | (° C.) | (° C.) | (° C.) |
| 14 | 92.9 | 87.5 | 27 | −2 | 25 |
| 15 | 96.0 | 94.1 | 37 | 2 | 39 |
| 16 | 92.0 | 102.2 | 29 | 16 | 45 |

*thermal transition at T_{g}2

Examples for Photosensitive Networks

10 mmol n-butylacrylate (BA), a cinnamic acid ester (0.1-3 mmol) and possibly 2 mmol hydroxyethylmethacrylate (HEMA) are mixed in a flask. 1 mol % AiBN and 0.3 mol % poly(propyleneglycol)dimethacrylate (Mₙ=560) are added to the mixture. The mixture is filled by means of a syringe into a mould of two silylated object carriers, between which a Teflon seal ring of a thickness of 0.5 mm is located. The polymerisation of the mixture takes place for 18 hours at 80° C.

The mould in which the cross linking takes place corresponds to the permanent mould. The mixture can also be cross linked in any other shapes.

After polymerization the network is removed from the mould and is covered by 150 mL hexane fraction. Subsequently, chloroform is gradually added. This solvent mixture is exchanged several times within 24 hours to solve out low-molecular and non cross linked components. Subsequently, the network is cleaned by means of hexane fraction and is dried over night in a vacuum at 30° C. The weight of the extracted sample relative to the preceding weight corresponds to the gel content. The two following tables show the quantities of the monomers used as well as the moisture expansion in chloroform and the gel content G thereof.

| | Monomer content of the mixture (mmol) | | | | | | | |
|---|---|---|---|---|---|---|---|---|
| Nr. | BA | HEMA-CA | HEA-CA | HPMA-CA | HPA-CA | PEGMA-CA | Q (%) | G (%) |
| 1A | 10 | 0.25 | — | — | — | — | 720 | 97.2 |
| 1B | 10 | 0.5 | — | — | — | — | 550 | 94.9 |
| 1C | 10 | 1 | — | — | — | — | 400 | 91.6 |
| 2A | 10 | — | 0.1 | — | — | — | 620 | 89.0 |
| 2B | 10 | — | 0.25 | — | — | — | 900 | 96.2 |
| 2C | 10 | — | 0.5 | — | — | — | 680 | 95.7 |
| 2D | 10 | — | 1 | — | — | — | 1320 | 96.5 |
| 2E | 10 | — | 2 | — | — | — | 1320 | 96.5 |
| 3A | 10 | — | — | 0.25 | — | — | 950 | 98.7 |
| 3B | 10 | — | — | 0.5 | — | — | 650 | 93.4 |
| 3C | 10 | — | — | 1 | — | — | 450 | 98.4 |
| 4A | 10 | — | — | — | 0.25 | — | 830 | 95.9 |
| 4B | 10 | — | — | — | 0.5 | — | 700 | 98.1 |
| 4C | 10 | — | — | — | 1 | — | 550 | 94.3 |
| 5A | 10 | — | — | — | — | 0.25 | 600 | 98.2 |
| 5B | 10 | — | — | — | — | 0.5 | 550 | 97.3 |
| 5C | 10 | — | — | — | — | 1 | 530 | 92.4 |

BA = butylacrylate; cinnamic acid ester: CA = cinnamic acid; HEMA = hydroxyethylmethacrylate; HEA = hydroxyethylacrylate; HPMA = hydroxypropylmethacrylate; HPA = hydroxypropylacrylate; PEGMA = poly(ethyleneglycol)methacrylate

In a further series, a portion of 2 mmol hydroxyethylmethacrylate (HEMA) is additionally added to the binary polymer systems, since by this comonomer a further possibility of controlling the mechanical properties of the polymer networks can be expected.

| | Monomer content of the mixture (mmol) | | | | | | | | |
|---|---|---|---|---|---|---|---|---|---|
| | | | HEMA- | HEA- | HPMA- | HPA- | PEGMA- | Q | G |
| Nr. | BA | HEMA | CA | CA | CA | CA | CA | (%) | (%) |
| 6A | 10 | 2 | 1 | — | — | — | — | 370 | 95.5 |
| 6B | 10 | 2 | 2 | — | — | — | — | 350 | 99.2 |
| 6C | 10 | 2 | 3 | — | — | — | — | 420 | 96.8 |
| 7A | 10 | 2 | — | 1 | — | — | — | 390 | 98.5 |
| 7B | 10 | 2 | — | 2 | — | — | — | 300 | 92.8 |
| 7C | 10 | 2 | — | 3 | — | — | — | 250 | 96.4 |
| 8A | 10 | 2 | — | — | 1 | — | — | 240 | 94.4 |
| 8B | 10 | 2 | — | — | 2 | — | — | 310 | 92.3 |
| 8C | 10 | 2 | — | — | 3 | — | — | 310 | 92.9 |
| 9A | 10 | 2 | — | — | — | 1 | — | 450 | 94.7 |
| 9B | 10 | 2 | — | — | — | 2 | — | 360 | 82.7 |
| 9C | 10 | 2 | — | — | — | 3 | — | 380 | 80.2 |
| 10A | 10 | 2 | — | — | — | — | 1 | 1300 | 83.4 |
| 10B | 10 | 2 | — | — | — | — | 2 | 1450 | 83.8 |
| 10C | 10 | 2 | — | — | — | — | 3 | 2150 | 84.8 |

Manufacture of the Inter-Penetrated Networks IPN

n-butylacrylate is cross linked with 3 percent by weight (0.6 mol %) poly(propyleneglycol)dimethacrylate (molecular weight 560 g/mol) in the presence of 0.1 percent by weight of AiBN, as described above. Subsequently, the film is welled in THF to solve out unused monomer, and is then dried again. Then the firm is welled in a solution of the star-shaped photo-reative macromonomer in THF (10 percent by weight) and is subsequently dried again. The charging of the network with the photo-reactive component is then approx. 30 percent by weight.

Manufacture of the Star-Shaped Photosensitive Macromonomers

Star-shaped poly(ethyleneglycol) with 4 arms (molecular weight 2000 g/mol) is solved in dry THF and triethylamine. For this purpose cinnamyliden acetylchloride slowly solved in dry THF is dripped. The reaction mixture is stirred for 12 hours at room temperature, then it is stirred for three days at 50° C. Fallen out salts are filtered off, the filtrate is concentrated and the product obtained is washed with diethylether. H-NMR measurements resulted in a conversion of 85%. From the UV-spectroscopic point of view, the macromonomer has an absorption maximum at 310 nm before photoreaction, after photoreaction it has an absorption maximum at 254 nm.

The polymer amorphous networks were examined in view of their further thermal and mechanical properties. The results of these examinations are combined in the following table.

| | | | Tensile | Breaking |
|---|---|---|---|---|
| | | E-module | strength | strain εᵣ |
| | T_{g} | E at RT | σᵣ at RT | bei RT |
| No. | (° C.) | (MPa) | (MPa) | (%) |
| 1A | −40.8 | 0.54 | 0.24 | 45 |
| 1B | −34.5 | 1.10 | 0.21 | 15 |
| 1C | −21.2 | 1.77 | 0.24 | 10 |
| 2A | −46.1 | 0.29 | 1.00 | 20 |
| 2B | −40.3 | 0.22 | 0.15 | 20 |
| 2C | −35.6 | 0.94 | 0.18 | 20 |
| 2D | −19.9 | 1.69 | 0.42 | 20 |
| 2E | −10.9 | 4.22 | 0.12 | 35 |
| 3A | −30.6 | 0.56 | 0.15 | 30 |
| 3B | −22.8 | 0.90 | 0.31 | 35 |
| 3C | −18.6 | 2.39 | 0.44 | 25 |
| 4A | −40.5 | 0.54 | 0.18 | 35 |
| 4B | −34.9 | 1.04 | 0.24 | 25 |
| 4C | −24.9 | 1.88 | 0.35 | 25 |
| 5A | −38.8 | 0.36 | 0.08 | 20 |
| 5B | −36.5 | 1.44 | 0.10 | 15 |
| 5C | −29.6 | 1.41 | 0.22 | 6 |
| 6A | −10.0 | 1.80 | 0.34 | 25 |
| 6B | 2.2 | 11.52 | 2.48 | 35 |
| 6C | 16.1 | 120.69 | 9.66 | 15 |
| 7A | −11.4 | 2.67 | 0.51 | 25 |
| 7B | 7.3 | 9.71 | 2.26 | 30 |
| 7C | 12.6 | 39.78 | 5.28 | 25 |
| 8A | −11.9 | 2.35 | 0.83 | 45 |
| 8B | 6.6 | 25.02 | 5.17 | 50 |
| 8C | 10.4 | 139.9 | 13.06 | 15 |
| 9A | 3.5 | 1.53 | 0.53 | 50 |
| 9B | 8.5 | 14.04 | 4.55 | 60 |
| 9C | 13.9 | 32.42 | 6.42 | 50 |
| 10A | −27.4 25.7 | 1.40 | 0.29 | 30 |
| 10B | −23.6 52.8 | 2.41 | 0.67 | 25 |
| 10C | −20.0 56.6 | 4.74 | 0.96 | 25 |
| 11A * | −46.5 | 0.15 | >1.60 | >2000 |
| 12A ** | −45.0 | 0.17 | 1.0-1.5 | 300-500 |
| before irradiation | | | | |
| 12A ** | −40.0 | 0.20 | 0.5-0.9 | 30-100 |
| after irradiation | | | | |

* network of n-butylacrylate; 0.3 mol % cross linking agent; without photo-reactive component
** IPN; 0.6 mol % cross linking agent, physically charged with photo-reactive component

The shape memory properties were determined in cyclical photo-mechanical experiments. For this purpose, punched-out, barbell-shaped sheet pieces having a thickness of 0.5 mm and a length of 10 mm and a width of 3 mm were used.

1. A stent, comprising an SMP material for use in the non-vascular or vascular field.

2. The stent as claimed in claim 1, wherein the stent comprises a basic structure of a material coated with an SMP material, preferably an SMP material with one of the following: one shape in the memory and two shapes in the memory.

3. The stent as claimed in claim 1, further comprising additional additives selected from among x-ray contrast substances and medically effective compounds.

4. The stent as claimed in claim 1, wherein the SMP material is selected from among the following: polymer networks, thermoplastic SMP materials, composite materials and blends.

5. The stent as claimed in claim 1, wherein the SMP material is selected from among at least one of SMP materials in which the SMP effect is induced thermally, it is photo-induced, the SMP material is biocompatible and the SMP material is haemocompatible.

6. The stent as claimed in claim 1, wherein the SMP material has at least one of the following:
values for e-module of 0.5 to 50 MPa, an elongation of break of 100% to 1200%, a reset fixation of more than 90%, preferably more than 92%, even more preferably more than 95%, and particularly preferably more than 98%, and a reset ratio after five cycles in the thermo-mechanical experiment of more than 90%, preferably more than 92%, even more preferably more than 95% and particularly preferably more than 98%.

7. A stent as claimed in claim 5, wherein the network includes at least one of the following: caprolacton units, pentadecalacton units, ethyleneglycol units, propyleneglycol units, lactic acid units and glycol acid units.

8. A stent as claimed in claim 6, wherein the network includes cross linked caprolactonmacromonomers.

9. A method of manufacturing a stent of a biodegradeable SMP material, comprising the processing of the SMP material to a stent by one of the following: extrusion methods, coating methods, metal casting methods and spinning and weaving methods.

10. A system comprising a stent of a biodegradeable SMP material, and including at least one of the following: a temperature-controlled balloon catheter and a balloon catheter with an optical fibre.

11. A method for the minimal invasive implantation of a stent, comprising the following steps:
placing a stent of SMP material onto a temperature-controlled balloon catheter or a balloon catheter with an optical fibre;
inserting the stent placed in this manner to the desired position;
heating the stent by inserting a heating medium into the catheter;
expanding the stent to carry out the programming of the SMP material;
inserting a cooling medium into the catheter to fix the stent in the expanded condition or introduction of light (preferably UV light) of a suitable wavelength to fix the stent in the expanded state;
removing the balloon catheter.

12. A method for removing an implanted stent of a biodegradeable SMP material, comprising:
inserting a balloon catheter into an implantation location;
inserting a heat medium into a balloon catheter to accomplish the following: heat the stent and to introduce light of a suitable wavelength;
activating the shape memory effect by heating or the effect of light so that the stent is transferred from its temporary shape into the permanent shape, and
removing the balloon catheter, together with the stent.

13. The method as claimed in claim 11, further comprising the step of introduction of a cooling medium after introducing the heating medium to cool the stent in the permanent shape, before removing same.

14. A method for the minimal invasive implantation of a stent, wherein the stent is an SMP material with a first shape in the memory and a second shape in memory, comprising:
placing the stent onto one of the following: a temperature-controlled balloon catheter and a balloon catheter with an optical fibre, wherein the SMP material exists in the first temporary shape in memory;
inserting the stent into a desired position,
heating the stent by inserting a heated medium into the catheter or introducing light of a suitable wavelength to obtain the second temporary shape in memory; and
removing the balloon catheter.

15. A method of removing an implanted stent, wherein the stent comprises an SMP material a first memory shape and a second memory shape, comprising:
inserting a balloon catheter into the implantation location,
inserting a heat medium into the balloon catheter to accomplish one of the following: heat the stent and introduce light of a suitable wavelength;
activating the shape memory effect by one of the following: the heating and the effect of light so that the stent is transferred from the second temporary shape in memory into a permanent shape; and
removing the balloon catheter, together with the stent.
